(19) United States
(12) Patent Application Publication
(10) Pub. No.: US 2024/0141419 A1
(43) Pub. Date: May 2, 2024

(54) PLASMID TARGET, PRIMER PROBE, KIT AND METHOD FOR DETECTING RESIDUAL HOST DNA IN CELLULAR OR VIRAL FORMULATION

(71) Applicant: Keshihua (Nanjing) Biotechnology Co., Ltd, Nanjing, Jiangsu (CN)

(72) Inventors: Shiping JIAO, Nanjing (CN); Binwen Ge, Ninjing (CN)

(21) Appl. No.: 18/351,651

(22) Filed: Jul. 13, 2023

Related U.S. Application Data
(63) Continuation of application No. PCT/CN2022/127776, filed on Oct. 26, 2022.

(51) Int. Cl. C12Q 1/6851; C12Q 1/6853

(52) U.S. Cl. CPC C12Q 1/6851; C12Q 1/6853; C12Q 2600/166

(57) ABSTRACT

The present application relates to the field of biotechnology, particularly to a plasmid target, a primer probe, a kit and a method for detecting residual host DNA in a cellular or viral formulation. The plasmid target is one or two selected from a plasmid Ori and a KanR gene, the Ori element has a gene sequence of SEQ ID NO. 1, and the KanR gene has a gene sequence capable of encoding a Kan R peptide chain, wherein the Kan R has amino acid sequences of SEQ ID NOs. 2-5. The present application also relates to a primer probe for the plasmid target, a kit, and a applying method.

### CROSS-REFERENCE TO RELATED APPLICATION

This application is a continuation of PCT application serial no. PCT/CN2022/127776, filed on Oct. 26, 2022. The entirety of PCT application serial no. PCT/CN2022/127776 is hereby incorporated by reference herein and made a part of this specification.

### REFERENCE TO AN ELECTRONIC SEQUENCE LISTING

The contents of the electronic sequence listing (SequenceListing.xml; Size: 13,775 bytes; and Date of Creation: Feb. 28, 2023) is herein incorporated by reference.

### TECHNICAL FIELD

The present application relates to the field of biotechnology, and, particularly, to a plasmid target, a primer probe, a kit and a method for detecting residual host DNA in a cellular or viral formulation.

### BACKGROUND ART

A cell and gene therapy has become the hottest technology in the field of biomedical medicine following antibody drugs. Based on the statistics from FDA, in recent years, there is a surge in number of cell and gene therapies that have entered an early clinical stage. It is expected that more than 200 IND applications will be received each year. As of Dec. 6, 2021, FDA had approved a total of 22 cell and gene therapy products.

There are a variety of viral vectors used for cell and gene therapies, including adenoviruses, lentiviruses, adeno-associated viruses, etc. Most of these vectors are produced by using HEK293T cells, in which residual DNA will be left as an impurity in a final vector product. Regulatory authorities in all countries have made corresponding provisions on the limit of host residual DNA. For example, it is indicated in Chinese Pharmacopoeia Version 2020 that residual amount of process-related impurities such as DNA of host cells should be detected and controlled at an acceptable level [Overview of Human Gene Therapy Products; Chinese Pharmacopoeia (Volume III) Version 2020]. In Technical Guidelines for Pharmaceutical Research and Evaluation of Gene Transduction and Modification Systems (Exposure Draft) issued by the Pharmaceutical Department of Biological Products of Center for Drug Evaluation, NMPA in 2020, an applicant is suggested to adopt a limit of residual DNA in non-tumorigenic cells less than 10 ng/dose.

In CAR-T and TCR-T cell therapies using viruses as vectors, it is difficult to detect an accurate value of residual host cell DNA (HCD) due to the fact that both of the final cellular formulation and HEK293T are human DNA and there is a low proportion of residual host cell DNA (HCD) relative to the amount of total DNA in the final cellular formulation. A currently adopted method still involves detecting the residual host DNA in a viral formulation, and ensuring no excessive HCD in the cellular formulation by strictly controlling the HCD amount in the viral formulation. However, the process exploration is cumbersome and costly due to too strict control of the HCD in the viral formulation.

Thus, there remains a need in the art for a means to accurately and conveniently detect residual host DNA in a cellular formulation.

### SUMMARY

In view of this, the present application provides a means for detecting residual host DNA in a formulation selected from a group consisting of a cellular formulation or a viral formation. In the present application, related processes are optimized in terms of residual host DNA of the final cellular or viral formulation, reducing excess process requirements and ultimately reducing costs. Specifically, according to the method of the present application, the residual host DNA (R1) is firstly detected in the viral formulation, and the extracellular DNA clearance rate during cell culture is calculated by detecting a plasmid residue (P1) in the viral formulation and a plasmid residue in the cellular formulation (P2), thereby calculating the residual host DNA in the cellular formulation: R2=R1*(P2/P1).

In a first aspect, the present application provides a plasmid target for detecting residual host DNA in a formulation selected from a group consisting of a cellular formation and a viral formulation, and the plasmid target is one or two selected from a group consisting of a plasmid Ori element and a KanR gene. In some embodiments, the Ori element has a gene sequence of SEQ ID NO. 1, and the KanR gene has a gene sequence capable of encoding a Kan R peptide chain. In some further embodiments, the KanR gene has gene sequences of SEQ ID NOs. 2-5. In some other embodiments, in addition to the gene sequences disclosed by the present application, the KanR gene may also be other derivative sequences capable of encoding a Kan R peptide chain, the similarity of which is maintained above 80%, preferably above 85%, more preferably above 90%, more preferably above 95%, still more preferably above 99% to the gene sequences disclosed by the present application. In some embodiments, plasmids containing Ori elements such as PUC19, PUC18, and PMD18-T may be used as a vector. In some embodiments, the above-mentioned primer probes may be labeled with fluorophores such as 5′ HEX, 3′ BHQ1; 5′ VIC, 3′ BHQ1; 5′ TAMRA, 3′ BHQ2; 5′ ROX, 3′ BHQ3; 5′ CYS, 3′ BHQ2 or 5′ CYS, 3′ BHQ3, etc.

In some embodiments, the cellular formulation is selected from a group consisting of TCR-T, CAR-T, CAR-NK, and CAR-M cellular formulations, and the viral formulation is selected from a group consisting of lentiviruses and adenoviruses.

In a second aspect, the present application provides a primer probe of the plasmid target for detecting residual host DNA in a formulation selected from a group consisting of a cellular formulation and a viral formulation according to the first aspect, in which the primer probe is one selected from a group consisting of SEQ ID NO. 6, SEQ ID NO. 7, and SEQ ID NO. 8 for a plasmid Ori element; and the primer probe is one selected from a group consisting of SEQ ID NO. 9, SEQ ID NO. 10, and SEQ ID NO. 11 for a plasmid KanR gene.

In a third aspect, the present application provides a kit for detecting residual host DNA in a formulation selected from a group consisting of a cellular formulation and a viral formulation, including the primer probe according to the second aspect.

In a fourth aspect, the present application provides a method for detecting residual host DNA in a formulation selected from a group consisting of a cellular formulation and a viral formulation including the following steps: detecting residual host DNA in the viral formulation (R1), calculating an extracellular DNA clearance rate during cell culturing by detecting plasmid residue in the viral formulation (P1) and detecting plasmid residue in the cellular formulation (P2) using the primer probe described in the second aspect, and then calculating the residual host DNA in the cellular formulation according to the following formula: R2=R1*(P2/P1).

In some embodiments, the residual host DNA in the viral formulation (R1) is detected by using a DNA residue detection Kit.

In some embodiments, detecting the plasmid residue in the viral formulation (P1) using the primer probe as described in the second aspect includes the following steps: 1) designing a primer; 2) preparing a quantitative standard product containing Ori and/or KanR elements by using a plasmid vector; 3) preparing a primer probe of the Ori or KanR elements into a primer mixture, adding an amplification mixture into the primer mixture to perform an amplification, and obtaining an amplification standard curve; and 4) calculating the plasmid residue in the viral formulation (P1) from the amplification standard curve.

In some embodiments, the plasmid residue in the cellular formulation is detected by using primers for Ori or KanR elements.

In summary, the present application can achieve at least one of the following beneficial technical effects:

1. A method for detecting residual host DNA in a final cellular or viral formulation of a cellular therapy is provided. According to the ratio of the detected residual plasmids in the viral vector and final cellular formulation, the clearance rate of residual DNA in the process from viral vector to final cellular formulation is calculated. The residual host DNA in the final cellular formulation is calculated according to the DNA clearance rate and the detection on the host DNA content of viral vector. This achieves the detection of residual host DNA in the final cellular or viral formulation, and solves the problem of a large difference between a detection value and an actual value in the detection of host residue at a virus vector end.

2. A method for detecting a plasmid residue is provided; effective detection targets (Ori and KanR elements) are provided; and an accurate detection amplification system is provided.

### BRIEF DESCRIPTION OF THE DRAWINGS

FIG. 1 shows a general process flow diagram for producing CAR-T and TCR-T cell therapy formulations in related technologies;

FIG. 2 shows a standard curve amplification plot of an Ori primer system in Example 1;

FIG. 3 shows a standard curve amplification plot of a KanR primer system in Example 1;

FIGS. 4-8 show the results of flow cytometry analysis of Jurkat cells infected with 100 μL of virus diluted for 20-fold, 540-fold, 1620-fold, and 4860-fold in Example 2;

FIG. 9 shows the detection results of a plasmid residue for virus diluted for 10-fold, 100-fold, 1000-fold, 10000-fold, and 100000-fold in Example 2;

FIG. 10 shows an amplification plot of plasmid residue in the mouse PBMC cellular formulation in Example 2;

FIG. 11 shows the results of flow cytometry analysis of a final cellular formulation of mouse PBMC cellular formulation in Example 3;

FIG. 12 shows an amplification plot of the plasmid residue in the viral vectors detected with the KanR primers in Example 3;

FIG. 13 shows an amplification plot of the plasmid residue in the final cellular formulation detected with the KanR primer system in Example 3;

FIG. 14 shows an amplification plot of the plasmid residue in the lentivirus detected with the Ori primers in Example 4;

FIG. 15 shows an amplification plot of the plasmid residue in the lentivirus detected with the Kan primers in Example 4;

FIG. 16 shows the results of flow cytometry analysis of the CD3 cellular formulation in Example 4;

FIG. 17 shows an amplification plot of the plasmid residue in the final cellular formulation detected with the Ori primer system in Example 4; and

FIG. 18 shows an amplification plot of the plasmid residue in the final cellular formulation detected with the KanR primer system in Example 4.

### DETAILED DESCRIPTION

In order to make the objects, technical solutions, and advantages of the embodiments according to the present application clearer, the technical solutions in the embodiments of the present application will be clearly and completely described below with reference to the drawings of the present application. Obviously, it will be appreciated by one of ordinary skilled in the art that the described embodiments are part of, but not all of the embodiments of the present application. All other embodiments obtained by a person of ordinary skilled in the art based on the embodiments of the present application without creative efforts shall fall within the protection scope of the present application.

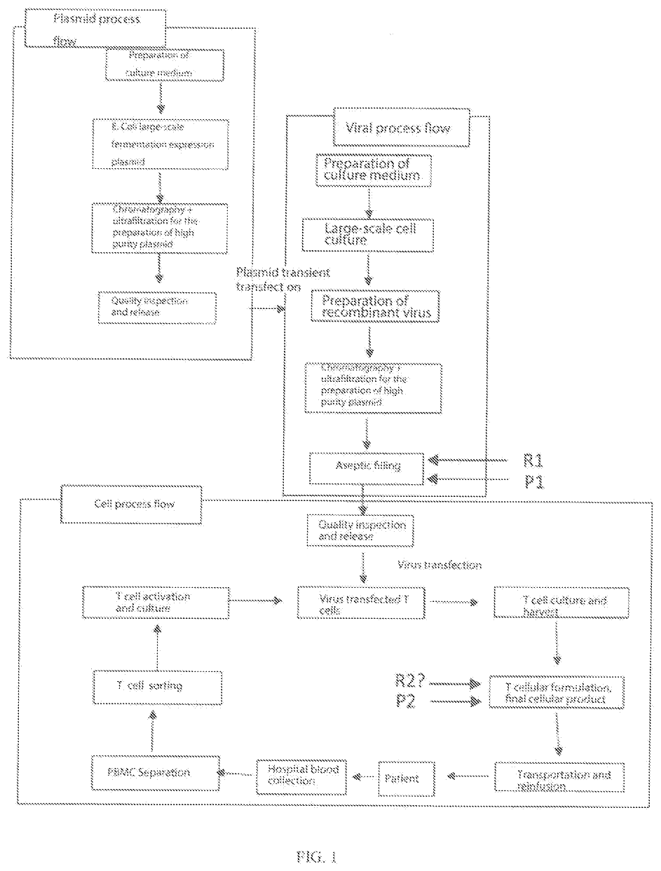

The main steps for producing CAR-T and TCR-T cell therapy formulations include initial isolation and enrichment of T cells, T cell activation, CAR/TCR gene transfer using viral or non-viral vector systems, T cell expansion in vitro, and final terminal processes and cryopreservation, as detailed in the process flow diagram of FIG. 1. In this process, the process of transferring the viral vector to the final cellular formulation may include exchange of cell medium or host cell DNA degradation during cell culture. The residual host DNA in the virus will be higher than that in the final cellular formulation, so the detection values cannot reflect an actual residual host DNA. In the Pharmacopeia, there is only requirements on the residual host DNA in the final cellular formulation, therefore, when the residue value in the virus vector is higher than that as required, the content of the host DNA can be only reduced in the virus process. Reduction of the residual host DNA in the viral vector requires elaborate process steps, such as degradation by addition of DNase. However, with the addition of excessive amount of the DNase, the problem of excessive DNase residue is caused, and the difficulty of subsequent viral vector purification process is increased, which will bring great challenges to the process requirements and cost. At the same time, in the process of preparing a viral formulation, the HCD value is required to meet the requirements of cellular formulation, which, however, is too severe, since residual DNA has to be removed with medium exchange and other operations during the process of adding virus to cells for transduction and cell culture.

Therefore, if a detection method that can accurately calculate the clearance rate can be developed and the accurate HCD value in the cellular formulation can be obtained, it will be of great significance for evaluating whether a cellular formulation meets the requirements of the Pharmacopoeia and guiding the exploration of virus process, as well as formulating a quality standard of a virus preparation.

In this regard, the inventor of the present application have conducted numerous studies. During the study, the present inventor recognized that, both of the finished cellular formulation TCR T and the viral residual host DNA are of human, so that it is impossible to identify whether the DNA extracted from the cellular formulation is the residual host DNA introduced by the addition of the virus or is originated from the TCR T cellular formulation itself. Meanwhile, during the study, the present inventors found that the virus had the same reduction ratio, i.e., clearance rate, as the residual plasmid and residual host DNA in the final cellular formulation during the process of transferring the virus vector to the final cellular formulation. Thus, the clearance rate of the residual host DNA from cellular process engineering can be calculated by detecting the plasmid residue in the final cellular formulation and the viral vector and comparing the ratio of the two plasmid residues. The amount of residual host DNA in the final cellular formulation can then be determined based on the amount of residual host DNA in the viral vector and the DNA clearance rate, as illustrated below.

| Detection sample | Detection target | |
|---|---|---|
| Viral vector | Plasmid residue | residual host DNA |
| Cell process such as | Plasmid residual | residual host DNA |
| virus transfection and T cell | reduction | reduction |
| expansion | | |
| Final cellular formulation | Plasmid residue | residual host DNA |

The plasmid and host DNA have the same DNA clearance rate during the cellular process, therefore, from the viral vector to the final cellular formulation, the plasmid residue and residual host DNA have the same residual proportion. Calculation formula: residual host DNA=Plasmid residue in a final cellular formulation/Plasmid residue in a viral vector*residual host DNA in a plasmid vector.

That is, the residual DNA clearance efficiency can be calculated by detecting the non-human plasmid residue at the viral stage and the plasmid residue at the cellular formulation stage, thereby indirectly calculating the residual host DNA in the cell formulation ca be indirectly calculated. On this basis, the inventors of the present application makes the present invention.

In particular, the present application provides a method for detecting the residue of residual host DNA in a cellular formulation. In this method, related processes are optimized by means of the residual host DNA of the final cellular formulation, so as to reduce excessive process requirements and ultimately reduce costs. In particular, a residual host DNA (R1) is firstly detected in a viral formulation, and an extracellular DNA clearance rate during cell culture is calculated by detecting the plasmid residue in the viral formulation (P1) and the plasmid residue in the cellular formulation residue (P2). Then, a residual host DNA in the cellular formulation is calculated by R2=R1*(P2/P1). In this way, the residual rate of DNA in the cellular formulation can be detected quickly and accurately. In addition, due to accurate detection of the residual host DNA in the final cellular formulation, the product can be used as a guide for better guiding a virus process and a cell process under the premise of meeting the requirements of the Pharmacopoeia.

The present application will be described in further details with reference to Examples hereinafter. It will be understood by those skilled in the art that these examples are given solely for purposes of facilitating their understanding and practice of the present application and are not intended to limit the scope of the present application to these particular examples.

### Example 1

### Detection of a Plasmid Residue Using Ori and KanR Elements on Plasmids as Targets

1) Primer Designing

As shown in Table 1, detection primer probes were designed for Ori (SEQ ID NO. 1) and KanR (SEQ ID NO. 3), respectively:

TABLE 1
Detection primer probes for Ori
(SEQ ID NO. 1) and Kan R (SEQ ID NO. 3)

| | Serial | Primer | Flourescent |
|---|---|---|---|
| Target | No. | probe | label |
| Ori-F | SEQ ID NO. 6 | GTTTGCCGGATCA | |
| | | AGAGCTAC | |
| Ori-R | SEQ ID NO 7 | GGCCTAACTACGG | 5′FAM |
| | | CTACACT | 3′BHQ1 |
| Kan-F | SEQ ID NO. 9 | CGCAATCACGAAT | |
| | | GAATAACGGT | |
| Kan-R | SEQ ID NO. 10 | CCAGACTTGTTCA | |
| | | ACAGGCCA | |
| Kan-P | SEQ ID NO. 11 | ACGCTCGTCATCA | 5′FAM, |
| | | AAATCACTCGCAT | 3′BHQ1 |

Primers were synthesized at Sangon Biotech (Shanghai) Co., Ltd., followed by HPLC purification.

2) Preparation of a Quantitative Standard

A quantitative standard containing targets Ori and Kan elements was prepared by ligating the KanR (SEQ ID NO. 3) sequence into the PUC57 vector carrying the Ori element. Plasmids were extracted and then detected for concentration using Nanodrop 2000 (Thermofisher, USA). 1 μg of plasmids were digested and linearized with restriction endonuclease QuickCut™ EcoR I (Takara Biotechnology (Dalian) Co., Ltd., Cat. No. 1611). According to the amount of the added digested plasmid and the molecular weight of the plasmid, the plasmid was diluted to a concentration of 3E8 copies/μL, and packaged by 100 μL/each bottle to prepare a plasmid standard.

3) qPCR Amplification System and Detection

Primer probes for the Ori or KanR genes were prepared into 20× Primer MIX mixtures. Concentrations of forward primers, reverse primers, and probes in the mixture were 8 μM, 8 μM, and 4 μM, respectively.

The amplification mixture was Premix Ex Taq™ (Probe qPCR) (Takara Biotechnology (Dalian) Co., Ltd., Cat. No. RR390A). 20 μL reaction system was prepared with Ori and Kan primers, respectively. Particular amounts of sample as added can be found in Table 2.

TABLE 2
Preparation of amplification system

| Component | Amount added/μL |
|---|---|
| Premix Ex Taq ™ (Probe qPCR) | 10 |
| 20× Primer MIX | 1 |
| Nuclease-free water | 6 |

10 μL of the plasmid standard in step 2) were diluted by adding 90 μL EASY Dilution (for Real Time PCR) (Takara Biotechnology (Dalian) Co., Ltd., Cat. No. 9160) to prepare 3E7 copies/μL solution. The solution was subjected to 10-fold gradient dilution to prepare STD0, STD1, STD2, STD3, STD4, and STD5 having concentrations of 3E6 copies/μL, 3E5 copies/μL, 3E4 copies/μL, 3E3 copies/μL, 3E2 copies/μL, and 30 copies/μL, respectively.

The diluted solution was aliquoted in a qPCR tube (96-well plate), 17 μL/well, with 3 μL standard being added in each well. Detection was performed using a QTOWER3G fluorescence quantitative PCR instrument (Analytik Jena AG, Germany). The amplification procedure was shown in Table 3 below.

TABLE 3
Amplification procedure

| | | Reaction | | Number of |
|---|---|---|---|---|
| Step | Temperature | time | | Cycles |
| Pre-denaturation | 95° C. | 3 | min | 1 |
| Denaturation | 95° C. | 10 | s | 40 |
| Extension | 60° C.* | 30 | s | |

*Collecting fluorescence signal (selecting fluorescence channel corresponding to the probe)

4) Amplification Results

The amplification results are shown in Table 4 below. As can be seen from Table 4, the amplification efficiencies of Ori and Kan primers were both greater than 0.9, the linear R2 was greater than 0.98, and the difference between the theoretical value of standard and the calculated concentration according to the standard curve was 80%-120%, which indicated that the primers and standards were well amplified.

TABLE 4
Amplification Results of Ori and Kan Primers

| Sample | Sample | | | Standard | Calculated | |
|---|---|---|---|---|---|---|
| Name | Type | Gene | Ct | concentration | concentration | |
| Amplification Results of Ori Primer | | | | | | |
| Std1 | Standard | ori | 19.59 | 300000 | 321611.3583 | R2 = 0.9987 |
| Std2 | Standard | ori | 23.04 | 30000 | 31304.09645 | Slope = −3.41 |
| Std3 | Standard | ori | 26.78 | 3000 | 2505.111032 | Intercept = 38.37 |
| Std4 | Standard | ori | 30.06 | 300 | 273.4954758 | Amplification |
| | | | | | | Efficiency = 0.9987 |
| Std5 | Standard | ori | 33.12 | 30 | 34.64092201 | |
| Amplification Results of Kan Primer | | | | | | |
| Std1 | Standard | Kan | 19.01 | 300000 | 278474.3995 | R2 = 0.9993 |
| Std2 | Standard | Kan | 22.13 | 30000 | 30741.8544 | Slope = −3.26 |
| Std3 | Standard | Kan | 25.27 | 3000 | 3346.107407 | Intercept = 36.76 |
| Std4 | Standard | Kan | 28.58 | 300 | 322.9999492 | Amplification |
| | | | | | | Efficiency = 0.9993 |
| Std5 | Standard | Kan | 32.10 | 30 | 26.88115052 | |

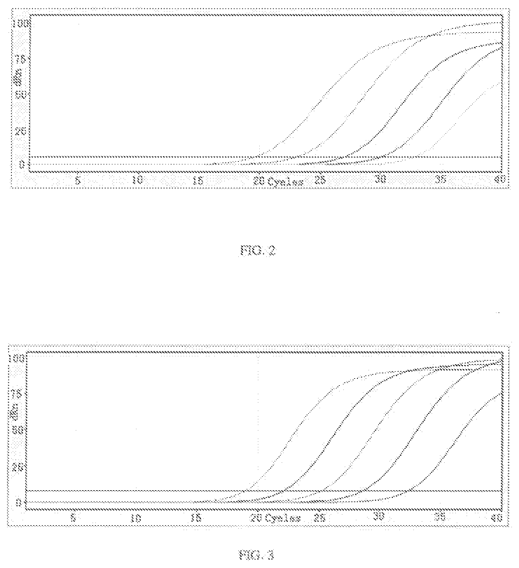

An amplification plot of the standard curve for the Ori primer system was shown in FIG. 2. An amplification plot of the standard curve for the Kan primer system was shown in FIG. 3.

5) Limit of Quantitation

The Ori and Kan primers were used to amplify the plasmid with STD5 concentration, respectively, and the amplification was repeated 20 times. It is showed that the amplifications were all successful, and the CV value was less than 10%, indicating that the detection limit of quantitation was less than 30 copies/μL.

### Example 2

### Method Validation: Comparison of Residual Host DNA Detection and Plasmid Residue Calculation in a Mouse TCR-T Final Cellular Formulation

The purpose of this example was to demonstrate that the method according to the present application is able to truly reflect the residual host DNA values in a cellular formulation, and to demonstrate that the method was reliable and accurate. The idea of verification was as follows: based on the production process of human TCR T cellular formulation, the human PBMC in the initial charge was replaced with murine PBMC, virus (accurately measuring the residual host DNA (R1) and plasmid residue (P1) therein) was added, and production was performed by the same process for T cell, including activation, amplification, perfusion, and washing, to obtain final murine TCR-T cells. At this time, the cellular formulation was able to accurately detect both the plasmid residue (P2) and the human residual host DNA (R2-TRUE). The calculated R2 according to P2/P1*R1=R2 was then compared with the actually measured R2-TRUE. 1. Production of mouse TCR T by simulated human TCR T process:

1) Initial Virus Titer and Measurements of R1 and P1

Virus was packaged by co-transfecting HEK293T cells using a four-plasmid system (packaging plasmids pMD2. G (Addgene Plasmid #12259), pRSV-Rev (Addgene Plasmid #12253), and pMDLg/pRRE (Addgene Plasmid #12251) purchased from Addgene, and the transfer plasmid was engineered pLenti.PGK.chFP.W (Addgene Plasmid #51008) to which the gene of target TCR was added). The infectious titers were detected by infecting Jurkat cells with the purified virus.

(1) Virus titer detection: Jurkat cell density was 5×10\hat{ }5 cells/mL, and cells were plated in a 24-well cell culture plate (1 mL per well); lentiviruses at −80° C. were taken out and thawed at 4° C. The lentiviruses were blended after the thawing, and diluted with DMEM medium. 100 μL virus diluent was added to each well, in which the virus diluent has 4 dilution gradients, and the dilution factor was 3. The diluent was blended by crossing, and incubated in 37° C., 5% CO₂ cell incubator for 48 h±2 h. The cultured cells were collected, labeled with antibody, and detected by flow cytometry. Virus titer was calculated based on the positive rate.

Selection of titer calculation data: the group with a positive rate of 1%-20% was selected for titer calculation;

calculation formula of titer: Titer (TU/mL)=N*P*D/V
where:
N=Number of cells before lentivirus infection;
P=cell positive rate (1%-20%);
V=Volume of lentivirus infected cells per well;
D=Dilution factor, 10-fold dilution, D=10; 100-fold dilution, D=100;
TU=Transduction unit.

The flow cytometry detection results are shown in Table 5 below, in which the virus titer is 2.54E+08 TU/ml.

TABLE 5
Flow cytometry detection results

| | | | | Average | |
|---|---|---|---|---|---|
| | Dilution | | Virus titer | titer | |
| Sample Name | factor | TCR-T+ | TU/mL | TU/mL | CV |
| Jurkat Blank | NA | 0.004 | NA | NA | NA |
| 1VVPD-211126 | 20 | 0.9347 | 3.67E+07 | NA | NA |
| | 540 | 0.1985 | 2.11E+08 | 2.54E+08 | 16.16% |
| | 1620 | 0.0813 | 2.59E+08 | | |
| | 4860 | 0.0306 | 2.92E+08 | | |

Note:
the result is effective only when the virus titer is calculated by taking the
positive rate of CAR from 1% to 20%.

| | | | |
|---|---|---|---|
| Cell count | 196000 | 1.97E+05 | 7.90% |
| | 182000 | | |
| | 213000 | | |

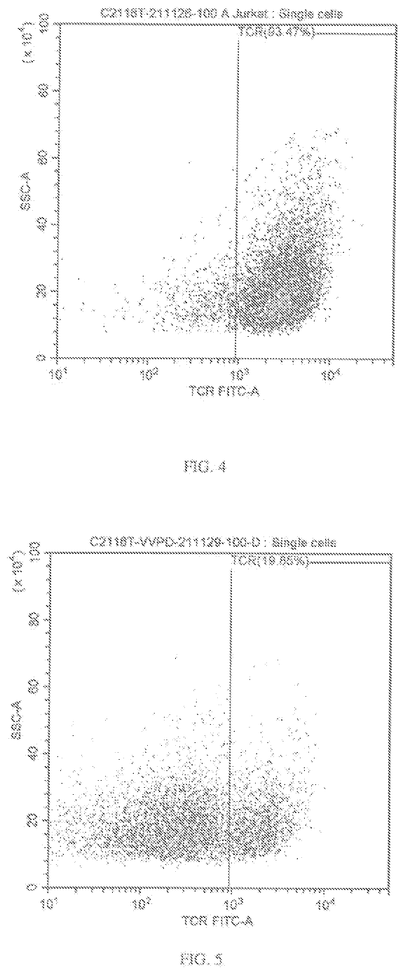

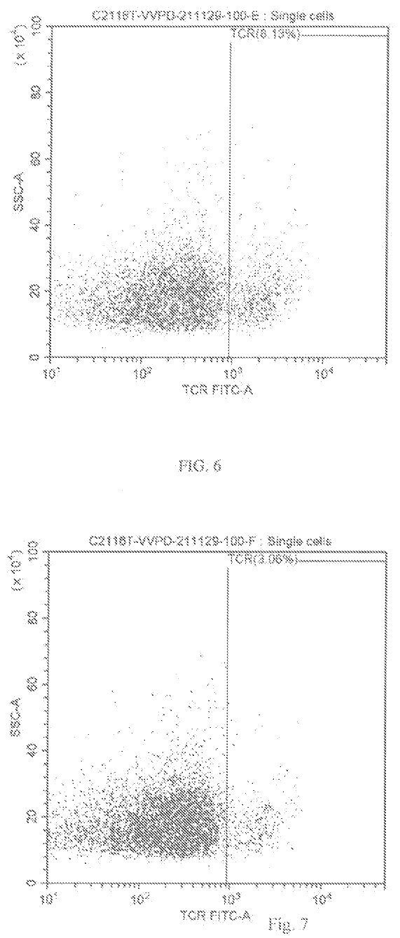

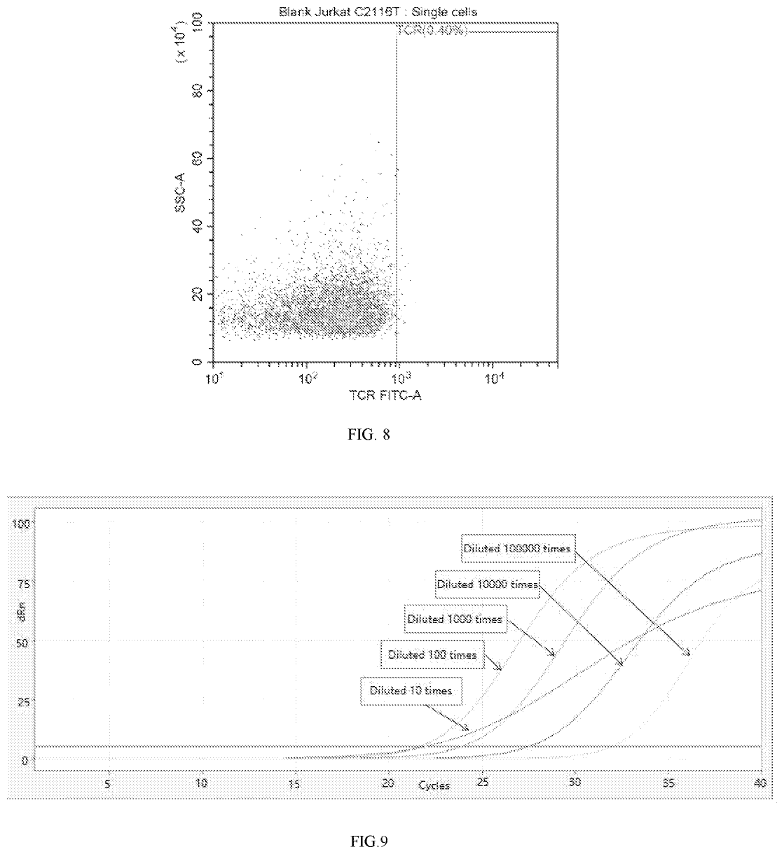

FIGS. 4-8 show the flow cytometry detection results of Jurkat cells infected with 100 μL of virus diluted by 20-fold, 540-fold, 1620-fold and 4860-fold, respectively.

(2) Detection of Host DNA in the Virus

50 μL of sample was extracted for DNA with Host Cell Residue DNA Sample Pretreatment Kit (magnetic bead method) (Cat. No. SK030203D100, Huzhou Shenke Biotechnology Co., Ltd.), and eluted with 50 μL eluent, with the other operations performed according to the instructions of the kit. The residual host DNA in the viral vector was detected with HEK293 Residual DNA Detection Kit (PCR-Fluorescent Probe Method) (Cat. No: 1101104). The detection value of host HEK293T DNA residue was 3.51E6 fg/μL by qPCR amplification.

(3) Detection of a Plasmid Residue in the Virus

The plasmid residue in the viral vector was detected by an Ori primer detection system, and an amplification system and an amplification standard curve were prepared as in Example 1. The virus treated sample was diluted with nuclease-free water in 10-fold gradient, and the plasmid residue results with the virus diluted by 10-fold, 100-fold, 1000-fold, 10000-fold and 100000-fold were detected, respectively. The detection results were shown in FIG. 6.

TABLE 6
Detection results of plasmid residue after virus dilution

| | Ct value | Concentration |
|---|---|---|
| 10-fold dilution | 21.14 | 1.13E5 copies/ml |
| 100-fold dilution | 20.93 | 1.30E5 copies/ml |
| 1000-fold dilution | 23.19 | 2.82E4 copies/ml |
| 10000-fold dilution | 26.80 | 2.47E3 copies/ml |
| 100000-fold dilution | 31.37 | 1.13E2 copies/ml |

As can be seen from FIG. 6, there was an inhibition on the amplification at 10-fold dilution. As the dilution factor was increased, the PCR-inhibiting component in the viral vector was diluted, and the detected valve was increased. However, as the dilution factor continued to increase, there was a loss for the sample. According to the detection results, a 1000-fold dilution is most suitable for the detection of the virus. The concentration of plasmid residue in the lentivirus was 2.82E7 copies/μL calculated from the dilution factor.

2) T Cell Activation and Viral Transduction

(1) Mouse CD4+/CD8+ Cell Isolation

127 immunodeficient mice OTI peripheral blood and spleen were collected, and PBMC were isolated under sterile conditions using mouse lymphocyte separation medium (Dakewe Biotech Co., Ltd., Cat. No. DKW33-R0100) according to the instructions. CD4+/CD8+ cells were sorted with CD4/CD8 (TIL) MicroBeads, mouse (Miltenyi, USA, Cat. No. 130-116-480) magnetic beads. Finally, 96 ml of CD4+/CD8+ cells were obtained at a cell density of 2E6/ml.

(2) Cell Activation

Cell activation was accomplished on a Sepax C-Pro Cell Processing System (Cytiva, USA) using the C-Pro CT-60.1 Processing Kit (Cytiva, USA). Cells were activated by resuspending the cells at 2E6 cell/ml in λ-VIVO medium (containing IL-2 200 IU/mL, CTS serum substitute 2%). 400 μL of Dynabeads™ Mouse T activator CD3/CD28 (Thermofisher, USA, Cat. No. 11456D) was added. After activation of 30 min in C-Pro, the activated cells were transferred to G-Rex®100M-CS (Wilson Wolf, USA, Cat. No. 81100-CS). G-Rex was transferred to an incubator at a condition of 37±1° C. and 5±0.5% CO₂ for culturing for 24±6 h.

(3) Virus Transduction

The lentivirus prepared in step (1) was thawed at room temperature. The thawed lentivirus was transferred to the cell counter for counting with a sampling bag, and the results were as follows: total cell density: 1.88E6/mL;
viable cell density: 1.68E6/mL;
total viable cells count: 1.68E8 (96 mL) 1.27 ml of 2.54E8 TU/ml virus was added at a multiplicity of infection (MOI) of 2:1.

The cell density was adjusted to 1E6 cell/ml by supplementing 64 ml X-VIVO medium (containing IL-2 200 IU/mL, CTS serum substitute 2%) (Lonza Biologics, USA, Cat. No. BE02-053Q). G-Rex cell culture device was gently circled horizontally and blended to disperse possibly aggregated cells at the bottom, and then the cells were transferred to an incubator at a condition of 37° C., 5% CO₂ for culturing for 48 h. The transduction process was completed.

3) T Cell Expansion Culture Perfusion and Washing Filling

GatheRex Liquid Handling (Wilson Wolf, USA, Cat. No. 80000E) was used to squeeze the liquid in the bottle of G-Rex cell culture device into the temporary cell storage bag for transduced cells. The temporary cell bag was placed on a magnetic board of a magnetic rack (CTS™ DynaMag™ Magnet, Cat. No. 12102, Thermo, USA) to remove the activated magnetic beads. After removing the activated magnetic beads, 10 L of the cell bag was placed on the matching tray, and the cell amplification system Xuri (Xuri Cell Expansion System W25, Cytiva, USA) was connected to a waste liquid bag (20-30 L). The adapter of the culture medium and the bag with the magnetic bead removed was connected with a Y-shaped adapter after being snap-locked, and was hung on the rack for liquid feeding. The parameters of Xuri W25 were set, the cell suspension and the culture medium were introduced into the Xuri cell bag through a peristaltic pump, so that the bag had a total volume of 1 L and a cell density of about 1E6/mL. After 8 days of perfusion culture, the culture volume reached 10 L and the cells were harvested. 3 ml of sample was taken from a sample bag and detected for the positive rate and cell count by flow cytometry. The viable cell density was 9.13E5 cell/ml and the total number of viable cells was approximately 1E10 cell. The positive rate of flow cytometry was 91%.

The amplified transduced T cells were harvested using a Sefia S-2000 cell harvester (Cytiva, USA) and its matched FlexCell program software. After concentration, washing, and freezing of the formulation, 350 ml of viable cells at a cell density of 2E7 cell/ml were obtained and dispensed into 7 cell cryopreservation bags, 50 ml/bag. Cell cryopreservation bags were cryopreserved in VIA Freeze Quad and then transferred to a liquid nitrogen reservoir.

4) T Cellular Formulation P2 and R2-TRUE Measurements

A bag of cryopreserved cells was detected for plasmid residue and residual host DNA. After the cellular formulation was thawed and blended, 50 μL of the cellular formulation was extracted for DNA with Host Cell Residue DNA Sample Pretreatment Kit (magnetic bead method) (Huzhou Shenke Biotechnology Co., Ltd., Cat. No. SK030203D100), and eluted with 50 μL of eluent, with the other operations performed according to the instructions. The extracted DNA was detected with Ori primer system for plasmid residue, and with Human Residual DNA Detection Kit (PCR-fluorescence probe method, Huzhou Shenke Biotechnology Co., Ltd.) for host 293 TDNA residue.

The plasmid residue in the viral vector was detected by an Ori primer detection system, and an amplification system and an amplification standard curve were prepared as in Example 1.

The detection value of plasmid residue was 1.87E3 copies/μL, and the results were shown in Table 7.

TABLE 7
Detection results of plasmid residue

| | Ct value | Concentration |
|---|---|---|
| Plasmid residue in the cellular | 27.22 | 1.87E3 copies/ul |
| formulation | | |

The mouse TCR-T cellular formulation prepared in step (3) was amplified for plasmid residue, and the amplification diagram of plasmid residue was shown in FIG. 10.

The residual host DNA of the cellular formulation prepared in step (3) was detected with Human Residual DNA Detection Kit (PCR-fluorescence probe method, Huzhou Shenke Biotechnology Co., Ltd.) and the detection result was 2.32E2 fg/μL.

5) Comparison of the calculated R2 according to P2/P1*R1=R2 with the actually measured R2-TRUE

1.27 ml of viral vector was used for transduction in step (1), and finally 350 ml of cellular formulation was obtained in step (3). The residual host DNA and plasmid residue in lentiviruses were introduced into the cells as virus transduction progresses, and both the plasmid residue and residual host DNA were reduced as the cells were expanded, perfused cultured, and finally washed to prepare a cellular formulation.

Amount of total plasmid residues in the cellular formulation P2/Amount of total plasmid residues in the lentivirus P1=1.83%

Amount of total residual host DNAs in the cellular formulation R2/Amount of total residual host DNAs in the lentivirus R1=1.82% From the calculation results, the residual proportion of the residual host DNA and

plasmid DNA residue was identical, indicating that the clearance rate of DNA residue was identical during the whole cell process. The residual host DNA in the cellular formulation can be calculated based on the residual proportion of plasmid in the cellular formulation and the residual host DNA in the lentivirus, i.e., R2=R1*(P2/P1). The comparison results were shown in Table 8 below.

TABLE 8
Comparison results of the residual amount

| | | Volume | |
|---|---|---|---|
| | | (virus dose/ | |
| | | cellular | |
| | Detection | formulation | |
| | value | volume) | Total amount |
| Plasmid residue | 2.82E7 | 1.27 ml | P1 = 2.82E7*1.27*1000 = |
| in lentivirus | copies/ul | | 3.58E10 copies |
| residual host | 3.51E6 | 1.27 ml | R1 = 3.51E6*1.27*1000 = |
| DNA in | fg/ul | | 4.45E9 fg |
| lentiviruses | | | |
| Plasmid residue | 1.87E3 | 350 ml | P2 = 1.87E3*350*1000 = |
| in cellular | copies/ul | | 6.55E8 copies |
| formulation | | | |
| residual host | 2.32E2 | 350 ml | R2-2.32E2*350*1000 = |
| DNA in cellular | fg/ul | | 8.12E7 fg |
| formulation | | | |

### Example 3

Mouse TCR-T Cells were Prepared Manually, and Detected and Compared for the Residual Proportion of the DNA Residue.

1. Isolation of Mouse PBMC

0.8 ml of immunodeficient mice OTI peripheral blood was drew, and PBMC were isolated under sterile conditions using mouse lymphocyte separation medium (Dakewe Biotech Co., Ltd., Cat. No. DKW33-R0100) according to the instructions.

2. 1E6 of the PBMC cells isolated in step 1 were seeded onto Advanced RPMI 1640 medium (Thermofisher, USA, Cat. No. 12633-012) containing 1 mL of 200 U/mL Recombinant mouse IL-2 (Thermofisher, USA, Cat. No. PMC0021) in a 24-well plate while 2 μL of Dynabeads™ mouse T activator CD3/CD28 (Cat. No. 11456D, Thermofisher, USA) was added, and the mixture was cultured in an incubator at a condition of 5% CO₂ at 37° C.

3. After 48 hours, the virus was added at a MOI of 2:1 based on cell count. The total number of cells was 7.5E5 and 5.9 μL of the viral vector used in Example 2 was added (infectious titer 2.54E8 TU/ml). After being cultured for 24 h in an incubator at a condition of 5% CO₂ at 37° C., the cells were centrifuged at 300 g, the supernatant was discarded, and the cells were resuspended in an Advanced RPMI 1640 medium (Thermofisher, USA, Cat. No. 12633-012) containing 1 mL of 200 U/mL Recombinant mouse IL-2 (Thermofisher, USA, Cat. No. PMC0021). The resuspended cells were further cultured in an incubator at a condition of 5% CO₂ at 37° C.

4. The cultured cells were counted every two days thereafter, and when the cells were proliferated to 2.5E6 cell s/mL, the cell density was controlled to 0.5E6/mL using Advanced RPMI 1640 medium supplemented containing 200 U/mL Recombinant mouse IL-2 (Thermofisher, USA, Cat. No. PMC0021). On the tenth day of virus transfection, the cells were harvested, centrifuged at 300 g, washed twice with PBS buffer (Thermofisher, USA, Cat. No. 10010-023), counted, and resuspended with cell cryopreservation solution (Dakewe Biotech Co., Ltd., Cat. No. UH-M1002-050) to obtain 8.5 mL of a final cellular formulation with a density of 1E7/mL. The positive rate detected by flow cytometry was 90.1%.

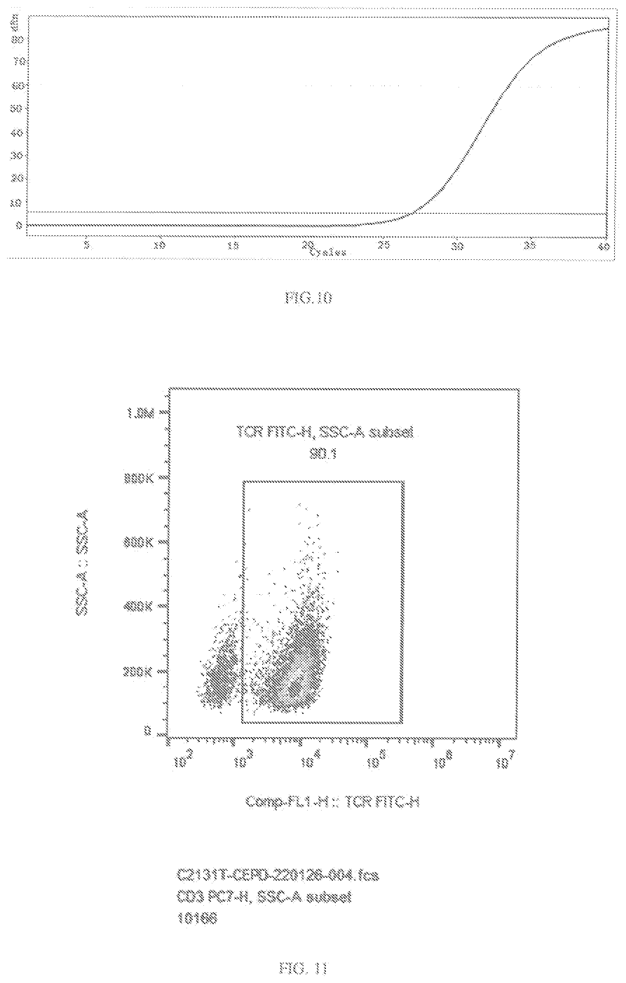

The flow cytometry detection results of the final cellular formulation were shown in FIG. 11, with a positive rate of 90.1%.

5. The plasmid residue in the viral vector was detected by Kan primers, and an amplification system and an amplification standard curve were prepared as in Example 1. The virus treated samples were diluted with nuclease-free water in a 10-fold gradient and diluted 1000-fold for detection.

Amount of total plasmid residues in the virus=concentration detected*dilution factor (1000)*volume of viral vector.

The calculation results of amount of total plasmid residues in the virus were shown in Table 9 below.

TABLE 9
Calculation results of amount of total plasmid residues in the virus

| | Viral vector (total amount of 5.9) | | |
|---|---|---|---|
| | | Concentration | Amount of total |
| | Ct | detected | plasmid residue |
| Plasmid residual KanR | 22.21 | 2.90E4 copies/ul | 1.71E8 copies |
| gene detection system | | | |

An amplification plot of the plasmid residue detected by the Kan primers for the viral vectors in this example was shown in FIG. 12.

50 μL of the cellular formulation was extracted for DNA with Host Cell Residue DNA Sample Pretreatment Kit (magnetic bead method) (Huzhou Shenke Biotechnology Co., Ltd., Cat. No. SK030203D100), and eluted with 50 μL of eluent, with the other operations performed according to the instructions. The plasmid residue in the final cellular formulation was detected by Kan primers, and an amplification system and an amplification standard curve were prepared as in Example 1. The detection results of amount of total plasmid residues were shown in Table below.

TABLE 10
Detection results of amount of total plasmid residues

| | Final cellular formulation (Total amount of 8.5 ml) | | |
|---|---|---|---|
| | | | Amount of total plasmid |
| | Ct | Concentration | residue |
| Plasmid residue | 25.49 | 2.86E3 | 2.43E7 copies |
| KanR Gene | | copies/μL | |
| detection system | | | |

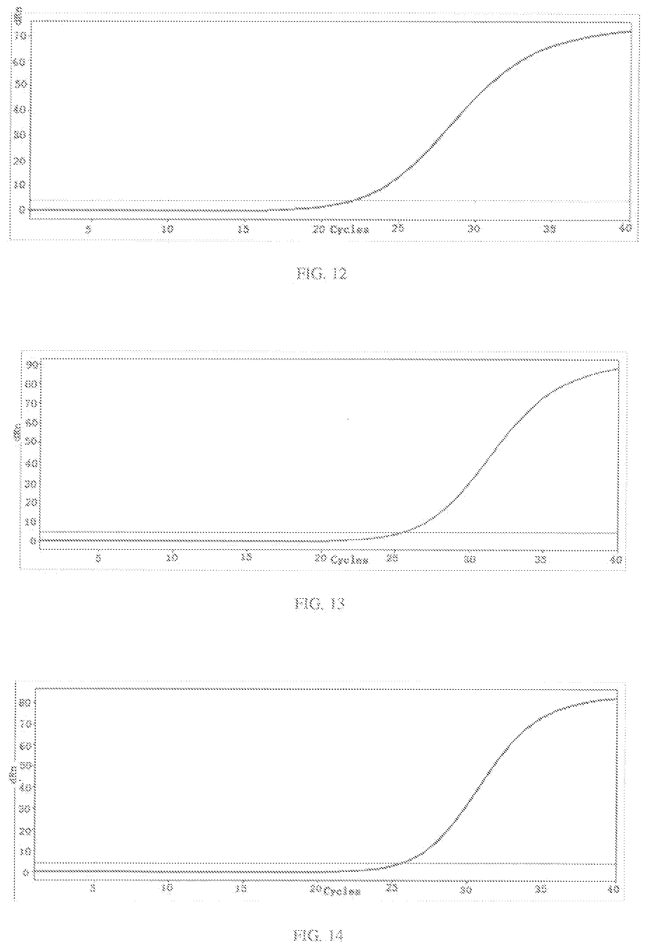

The amplification plot of the plasmid residue in the final cellular formulation detected by a Kan primer system was shown in FIG. 13.

6. The DNA extracted with Host Cell Residue DNA Sample Pretreatment Kit (magnetic bead method) (Huzhou Shenke Biotechnology Co., Ltd., Cat. No. SK030203D100) in step 5 was detected using Human Residual DNA Detection Kit (PCR-fluorescence probe method) (Huzhou Shenke Biotechnology Co., Ltd.) for the residual host DNA in the final cellular formulation. The detection value was 3.38E2 fg/μL.

The amount of total plasmid DNA residues in the virus P1=1.71E8 copies;

Amount of total plasmid DNA residues in the final cellular formulation P2=2.43E7 copies.

As can be seen from Example 2 that the amount of total residual host DNAs in the virus R1=3 0.51E6fg/ul*5.9 ul=2.07E7 fg;

Amount of total residual host DNAs in the final cellular formulation R2=3.38E2 fg/ul*8.5 ml=2.87E6 fg.

The ratio of the amount of total plasmid DNA residues in the final cellular formulation P2 to the amount of total plasmid DNA residues in the virus P1 (P2/P1=14.21%) was almost equal to the ratio of the amount of total residual host DNAs in the final cellular formulation R2 to the amount of total residual host DNAs in the virus R1 (R2/R1=13.86%), with a difference of less than 5%.

The above results indicated that the reducing proportions of the residual host DNA and the plasmid DNA residue were same after the steps of virus transduction, cell culture expansion, and washing.

The difference between the host DNA clearance rate in the process calculated according to the residual host DNA and the plasmid DNA clearance rate calculated according to the plasmid result was within 5%. It was indicated that the change of the plasmid DNA can reflect the change of host HEK293T DNA, and it was feasible to calculate the residual host DNA in the final cellular formulation based on the change of plasmid.

### Example 4

Detection and calculation of the residual host DNA of the final product in the actual production of TCR-T by applying this method (practical application in cellular formulation production)

1. Condition of Viral Formulation: Titer P1 R1

Final lentivirus product titer: 5.15×10⁸ TU/mL.

1) Detection of Host DNA in the Virus

50 μL of sample was extracted for DNA with Host Cell Residue DNA Sample Pretreatment Kit (magnetic bead method) (Huzhou Shenke Biotechnology Co., Ltd., Cat. No. SK030203D100), and eluted with 50 μL eluent, with the other operations performed according to the instructions of the kit. The residual host DNA in the viral vector was detected with Human Residual DNA Detection Kit (PCR-Fluorescent Probe Method).

The host HEK293T DNA residue was 3.67E4 fg/ul, as detected by qPCR amplification. 1) Detection of the plasmid residue in the virus

Ori primer system and Kan primer system were used to detect the plasmid residue in the viral vector, respectively. After 1000-fold dilution of the final virus product, the amplification system was prepared as in Example 1 and loaded for detection. The detection results of the amount of total viral vector were shown in Table 11 below.

TABLE 11
Detection results of the amount of total viral vector

| | Viral vector (total amount of 680 μL) | | |
|---|---|---|---|
| | Ct value | Concentration | Total amount P1 |
| Plasmid residue Ori | 25.12 | 7.63E3 copies/ul | 5.19E9 copies |
| Detection system | | | |
| Plasmid residue KanR | 23.88 | 8.91E3 copies/ul | 6.06E9 copies |
| gene detection system | | | |

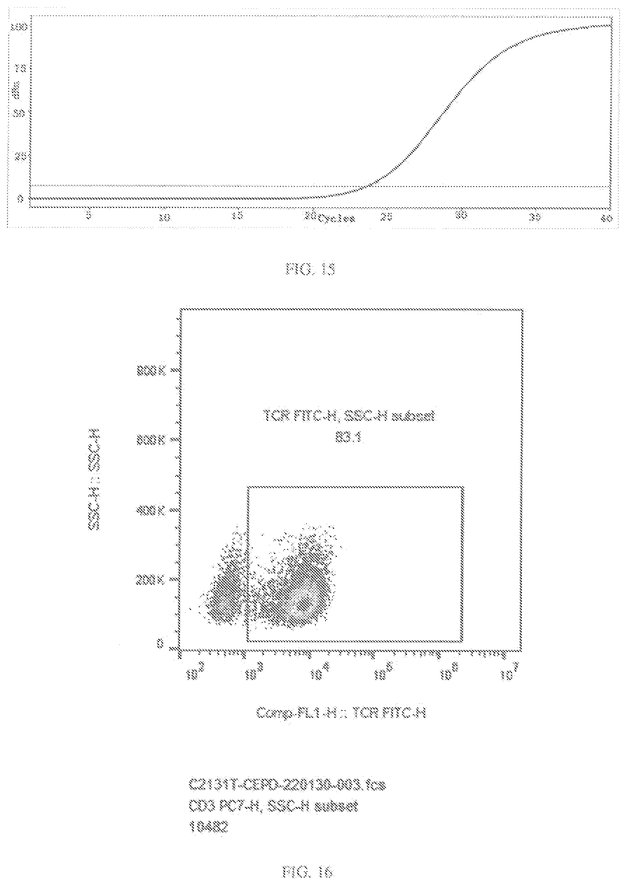

The lentivirus plasmid residue amplification plot detected by Ori primers was shown in FIG. 14. The lentivirus plasmid residue amplification plot detected by Kan primers was shown in FIG. 15.

2. Production Process of Cellular Formulation

1) T Cell Activation

CD3 cells were activated, and CD3/CD28 activated magnetic beads were added to the 3E8 CD3 positive cells (error range±10%), such that the ratio of CD3 cells to magnetic beads reached 1:1. The mixture was incubated 30 min in Sepax Cpro cell processor (Cytiva, USA) with a density of CD3 positive cells of 3E6 cells/mL. The cells were transferred to a G-rex cell culture device with a controlled activation density of 2E6 cells/mL. The G-rex cell culture device was transferred to an incubator at a condition of 37° C., 5% CO₂ for culturing 24 h to complete the activation process.

2) T Cell Transduction

Samples were taken from the G-Rex into a cell counter using a sampling bag, with the following results:
Total cell density: 1.63 E6/mL
Viable cell density: 1.16 E6/mL
Total viable cells count: 1.74 E8 (150 mL).

The lentivirus vectors were transferred to room temperature for being thawed and then 0.68 ml of lentivirus was added at a MOI=2, according to the viral titer report. The diluted virus vector solution was prepared by blending the quantitative lentivirus vector with the transduction medium.

The cell viral vector mixture was transferred via transfer tube to a G-rex cell culture device, and the density was adjusted to 1E6 cells/mL with X-VIVO (CTS serum substitute containing 2% of IL-2 200 IU/mL) medium. The G-rex cell culture device was transferred to an incubator at a condition of 37±1° C., 5±0.5% CO₂ carbon dioxide for culturing for 48±6 h to complete the transduction process.

The transduced cells were taken out from the carbon dioxide incubator, and the density was adjusted to 1E6 cells/mL with the culture medium. The G-rex cell culture device was transferred to an incubator at a condition of 37° C., 5% CO₂ carbon dioxide for culturing for 24 h.

3) Cell Expansion

GatheRex Liquid Handling (Wilson Wolf, USA, Cat. No. 80000E) was used to squeeze the liquid in the bottle of G-Rex bottle into the temporary cell storage bag for transduced cells. The temporary cell bag was placed on a magnetic board of a magnetic rack (CTS™ DynaMag™ Magnet, Cat. No. 12102, thermo, USA) to remove the activated magnetic beads. After removing the activated magnetic beads, 10 L of the cell bag was placed on the matching tray, and the cell amplification system Xuri (Xuri Cell Expansion System W25, Cytiva, USA) was connected to a waste liquid bag (20-30 L). The adapter of the culture medium and the bag with the magnetic bead removed was connected with a Y-shaped adapter after being snap-locked, and was hung on the rack for liquid feeding. The parameters of Xuri W25 were set, the cell suspension and culture medium were introduced into the Xuri cell bag through a peristaltic pump, so that the bag had a total volume of 1 L and a cell density of about 1E6/mL. The culture system was cultured from 1000 mL to 5000 mL, and the 5000 mL culture system was maintained until the number of CAR positive cells was more than or equal to 1.5E10 cells.

Amplified transduced T cells were harvested using a Sefia S-2000 cell harvester (Cytiva, USA) and its matched FlexCell program software. After concentration, washing, and freezing of the formulation, 352 ml of viable cells at a cell density of 5E7 cell/ml were obtained and dispensed into 7 cell cryopreservation bags, 50 ml/bag. Cell cryopreservation bags were cryopreserved in VIA Freeze Quad and then transferred to a liquid nitrogen reservoir. The cellular formulation was subjected to flow cytometry, with a positive rate of 83.1%. The detection results were shown in FIG. 16.

3. Calculated Values for Cellular Formulations P2 and R2

1) Detection of the Plasmid Residue in the Cellular Formulation

Ori primer system and KanR primer system were used to detect the amount of plasmid residue in the final cellular formulation, respectively. 50 μL of sample was extracted for DNA with Host Cell Residue DNA Sample Pretreatment Kit (magnetic bead method) (Cat. No. SK030203D100, Huzhou Shenke Biotechnology Co., Ltd.), and eluted with 50 μL eluent, with the other operations performed according to the instructions of the kit. Amount of total plasmid residues in the virus=concentration detected*dilution factor (1000)*volume of virus vector. Amount of total plasmid residues in the final cellular formulation=concentration detected*dilution factor (1)*volume of final cellular formulation. The detection results were shown in Table 12 below.

TABLE 12
Detection results of amount of total plasmid residues in the final
cellular formulation

| | Final cellular formulation | | |
|---|---|---|---|
| | (total amount of 352 mL) | | |
| | Ct value | Concentration | Total amount P2 |
| Plasmid residue Ori | 29.8 | 2.84E2 copies/ul | 1.00E8 copies |
| detection system | | | |
| Plasmid residue | 28.6 | 3.18E2 copies/ul | 1.12E8 copies |
| KanR gene | | | |
| detection system | | | |

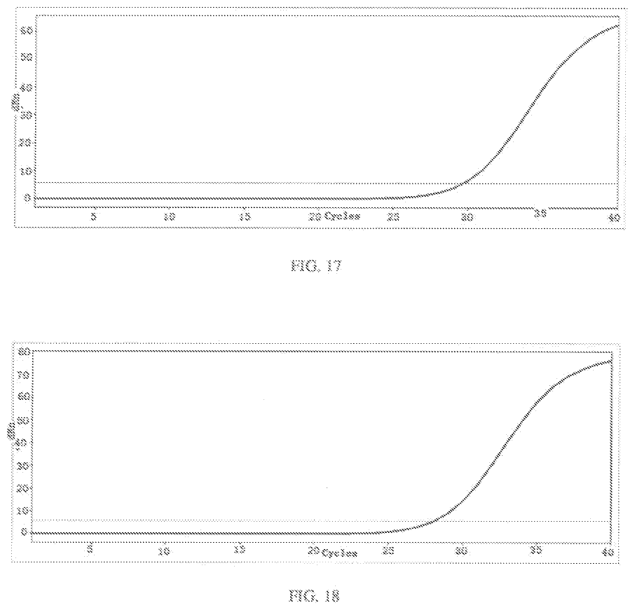

The amplification plot of the plasmid residue in the final cellular formulation detected by an Ori primer system was shown in FIG. 17. The amplification plot of the plasmid residue in the final cellular formulation detected by a Kan primer system was shown in FIG. 18.

According to the Ori detection results, the amount of total plasmid residues in the final cell product P2: the amount of total plasmid residues in lentivirus P1=1.93%;

According to the KanR gene detection results, the amount of total plasmid residues in the final cell product P2: the amount of total plasmid residues in lentivirus P1=1.85%;

The residual host DNA concentration in the lentivirus was 3.67E4 fg/ul, for a total of 680 μL of virus. The amount of total residual host DNAs in lentivirus R1 was 2.50E7 fg.

The amount of total residual host DNAs in the cellular formulation R2 was calculated to be 4.83E5 fg according to the formula R2=R1*(P2/P1) based on the detection results of Ori.

The amount of total residual host DNAs in the cellular formulation R2 was calculated to be 4.63E5 fg according to the formula R2=R1*(P2/P1) based on the detection results of KanR gene.

Residual host DNA in the viral vector was 25 ng, which exceeded the requirement of less than 10 ng/dose required by the Guidelines. The detected residual host DNA in the final cellular formulation was less than 1 ng, which was far below the limit requirements in the Guidelines. In the process from the virus vector to the final cellular formulation, different production processes resulted in different variations in the residual host DNA carried in the viral vectors. By detecting and comparing the amount of total plasmid residues in the cellular formulation and the amount of total plasmid residues introduced into the lentivirus, the residual host DNA in the final cellular formulation can be accurately evaluated.

The above descriptions are merely part of the embodiments of the present application. It should be noted that a person of ordinary skilled in the art may further make several improvements and modifications without departing from the principle of the present application, but such improvements and modifications should be deemed as falling within the protection scope of the present application.

gene sequence
SEQ ID NO. 1
TTGAGATCCTTTTTTTCTGCGCGTAATCTGCTGCTTGCAA
ACAAAAAAACCACCGCTACCAGCGGTGGTTTGTTTGCCGG
ATCAAGAGCTACCAACTCTTTTTCCGAAGGTAACTGGCTT
CAGCAGAGCGCAGATACCAAATACTGTTCTTCTAGTGTAG
CCGTAGTTAGGCCACCACTTCAAGAACTCTGTAGCACCGC
CTACATACCTCGCTCTGCTAATCCTGTTACCAGTGGCTGC
TGCCAGTGGCGATAAGTCGTGTCTTACCGGGTTGGACTCA
AGACGATAGTTACCGGATAAGGCGCAGCGGTCGGGCTGAA
CGGGGGGTTCGTGCACACAGCCCAGCTTGGAGCGAACGAC
CTACACCGAACTGAGATACCTACAGCGTGAGCTATGAGAA
AGCGCCACGCTTCCCGAAGGGAGAAAGGCGGACAGGTATC
CGGTAAGCGGCAGGGTCGGAACAGGAGAGCGCACGAGGGA
GCTTCCAGGGGGAAACGCCTGGTATCTTTATAGTCCTGTC
GGGTTTCGCCACCTCTGACTTGAGCGTCGATTTTTGTGAT
GCTCGTCAGGGGGGCGGAGCCTATGGAAA
amino acid sequence
SEQ ID NO. 2
SRPRLNSNMDADLYGYKWARDNVGQSGATIYRLYGKPDAP
ELFLKHGKGSVANDVTDEMVRLNWLTEFMPLPTIKHFIRT
PDDAWLLTTAIPGKTAFQVLEEYPDSGENIVDALAVFLRR
LHSIPVCNCPFNSDRVFRLAQAQSRMNNGLVDASDFDDER
NGWPVEQVWKEMHKLLPFSPDSVVTHGDFSLDNLIFDEGK
LIGCIDVGRVGIADRYQDLAILWNCLGEFSPSLQKRLFQK
YGIDNPDMNKLQFHLMLDEFF
KanR gene sequence 1
SEQ ID NO. 3
ATGAGCCATATTCAACGGGAAACGTCGAGGCCGCGATTAA
ATTCCAACATGGATGCTGATTTATATGGGTATAAATGGGC
TCGCGATAATGTCGGGCAATCAGGTGCGACAATCTATCGC
TTGTATGGGAAGCCCGATGCGCCAGAGTTGTTTCTGAAAC
ATGGCAAAGGTAGCGTTGCCAATGATGTTACAGATGAGAT
GGTCAGACTAAACTGGCTGACGGAATTTATGCCACTTCCG
ACCATCAAGCATTTTATCCGTACTCCTGATGATGCATGGT
TACTCACCACTGCGATCCCCGGAAAAACAGCGTTCCAGGT
ATTAGAAGAATATCCTGATTCAGGTGAAAATATTGTTGAT
GCGCTGGCAGTGTTCCTGCGCCGGTTGCACTCGATTCCTG
TTTGTAATTGTCCTTTTAACAGCGATCGCGTATTTCGCCT
CGCTCAGGCGCAATCACGAATGAATAACGGTTTGGTTGAT
GCGAGTGATTTTGATGACGAGCGTAATGGCTGGCCTGTTG
AACAAGTCTGGAAAGAAATGCATAAACTTTTGCCATTCTC
ACCGGATTCAGTCGTCACTCATGGTGATTTCTCACTTGAT
AACCTTATTTTTGACGAGGGGAAATTAATAGGTTGTATTG
ATGTTGGACGAGTCGGAATCGCAGACCGATACCAGGATCT
TGCCATCCTATGGAACTGCCTCGGTGAGTTTTCTCCTTCA
TTACAGAAACGGCTTTTTCAAAAATATGGTATTGATAATC
CTGATATGAATAAATTGCAGTTTCATTTGATGCTCGATGA
GTTTTTCTAA
KanR gene sequence 2
SEQ ID NO. 4
ATGAGCCATATTCAACGGGAAACGTCTTGCTCTAGGCCGC
GATTAAATTCCAACATGGATGCTGATTTATATGGGTATAA
ATGGGCTCGCGATAATGTCGGGCAATCAGGTGCGACAATC
TATCGATTGTATGGGAAGCCCGATGCGCCAGAGTTGTTTC
TGAAACATGGCAAAGGTAGCGTTGCCAATGATGTTACAGA
TGAGATGGTCAGACTAAACTGGCTGACGGAATTTATGCCT
CTTCCGACCATCAAGCATTTTATCCGTACTCCTGATGATG
CATGGTTACTCACCACTGCGATCCCTGGGAAAACAGCATT
CCAGGTATTAGAAGAATATCCTGATTCAGGTGAAAATATT
GTTGATGCGCTGGCAGTGTTCCTGCGCCGGTTGCATTCGA
TTCCTGTTTGTAATTGTCCTTTTAACAGCGATCGCGTATT
TCGTCTCGCTCAGGCGCAATCACGAATGAATAACGGTTTG
GTTGATGCGAGTGATTTTGATGACGAGCGTAATGGCTGGC
CTGTTGAACAAGTCTGGAAAGAAATGCATAAACTTTTGCC
ATTCTCACCGGATTCAGTCGTCACTCATGGTGATTTCTCA
CTTGATAACCTTATTTTTGACGAGGGGAAATTAATAGGTT
GTATTGATGTTGGACGAGTCGGAATCGCAGACCGATACCA
GGATCTTGCCATCCTATGGAACTGCCTCGGTGAGTTTTCT
CCTTCATTACAGAAACGGCTTTTTCAAAAATATGGTATTG
ATAATCCTGATATGAATAAATTGCAGTTTCATTTGATGCT
CGATGAGTTTTTCTAA
KanR gene sequence 3
SEQ ID NO. 5
ATGAGCCATATTCAACGGGAAACGTCGAGGCCGCGATTAA
ATTCCAACATGGATGCTGATTTATATGGGTATAAATGGGC
TCGCGATAATGTCGGGCAATCAGGTGCGACAATCTATCGC
TTGTATGGGAAGCCCGATGCGCCAGAGTTGTTTCTGAAAC
ATGGCAAAGGTAGCGTTGCCAATGATGTTACAGATGAGAT
GGTCAGACTAAACTGGCTGACGGAATTTATGCCTCTTCCG
ACCATCAAGCATTTTATCCGTACTCCTGATGATGCATGGT
TACTCACCACTGCGATCCCCGGAAAAACAGCATTCCAGGT
ATTAGAAGAATATCCTGATTCAGGTGAAAATATTGTTGAT
GCGCTGGCAGTGTTCCTGCGCCGGTTGCATTCGATTCCTG
TTTGTAATTGTCCTTTTAACAGCGATCGCGTATTTCGTCT
CGCTCAGGCGCAATCACGAATGAATAACGGTTTGGTTGAT
GCGAGTGATTTTGATGACGAGCGTAATGGCTGGCCTGTTG
AACAAGTCTGGAAAGAAATGCATAAACTTTTGCCATTCTC
ACCGGATTCAGTCGTCACTCATGGTGATTTCTCACTTGAT
AACCTTATTTTTGACGAGGGGAAATTAATAGGTTGTATTG
ATGTTGGACGAGTCGGAATCGCAGACCGATACCAGGATCT
TGCCATCCTATGGAACTGCCTCGGTGAGTTTTCTCCTTCA
TTACAGAAACGGCTTTTTCAAAAATATGGTATTGATAATC
CTGATATGAATAAATTGCAGTTTCATTTGATGCTCGATGA
GTTTTTCTAA

1. A plasmid target for detecting residual host deoxyribonucleic acid (DNA) in a formulation selected from a group consisting of a cellular formulation and a viral formulation, wherein the plasmid target is one or two selected from a group consisting of a plasmid Ori element and a KanR gene.

2. The plasmid target according to claim 1, wherein the plasmid Ori element has a gene sequence of SEQ ID NO. 1, and the KanR gene has a gene sequence configured to encode a KanR peptide chain.

3. The plasmid target according to claim 2, wherein the KanR gene has a gene sequence selected from a group consisting of SEQ ID NO. 2, SEQ ID NO. 3, SEQ ID NO. 4, and SEQ ID NO. 5.

4. The plasmid target according to claim 1, wherein the cellular formulation is selected from a group consisting of TCR-T, CAR-T, CAR-NK, and CAR-M cellular formulations, and the viral formulation is selected from a group consisting of lentiviruses and adenoviruses.

5. A primer probe of the plasmid target for detecting residual host DNA in the cellular formulation or the viral formulation according to claim 1, wherein the primer probe comprises sequences of SEQ ID NO. 6, SEQ ID NO. 7, and SEQ ID NO. 8 for the plasmid Ori element; and the primer probe comprises sequences of SEQ ID NO. 9, SEQ ID NO. 10, and SEQ ID NO. 11 for the KanR gene.

6. A method for detecting residual host deoxyribonucleic acid (DNA), in a cellular formulation and a viral formulation, comprising the following steps: detecting the residual host DNA in the viral formulation (R1), calculating an extracellular DNA clearance rate during cell culture by detecting a plasmid residue in the viral formulation (P1) and detecting a plasmid residue in the cellular formulation (P2) with a primer probe, and then calculating the residual host DNA in the cellular formulation (R2) according to the following formula: R2=R1*(P2/P1), wherein the primer probe comprises a sequence of SEQ ID NO. 6.

7. The method according to claim 6, wherein the residual host DNA in the viral formulation (R1) is detected using a DNA residue detection Kit.

8. The method according to claim 6, wherein detecting the plasmid residue in the viral formulation (P1) with the primer comprises the following steps:
1) designing a primer;
2) preparing a quantitative standard comprising of Ori targets with a plasmid vector;
3) preparing a primer probe of an Ori gene into a primer mixture, adding the primer mixture into an amplification mixture to perform an amplification, and obtaining an amplification standard curve; and
4) calculating the plasmid residue in the viral formulation (P1) in accordance with the amplification standard curve.

9. The method according to claim 6, wherein the plasmid residue in the cellular formulation (P2) is detected using primers for an Ori gene.


SEQUENCE LISTING (11 sequences)

SEQ ID NO: 1 - DNA, 589 bp, Plasmavirus L2
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 2 - protein, 261 aa, Plasmavirus L2
SRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 3 - DNA, 810 bp, Plasmavirus L2
  source 1..810: mol_type = other DNA
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 4 - DNA, 816 bp, Plasmavirus L2
  source 1..816: mol_type = other DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 5 - DNA, 810 bp, Plasmavirus L2
  source 1..810: mol_type = other DNA
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 6 - DNA, 21 bp, synthetic construct
  source 1..21: mol_type = other DNA
gtttgccggatcaagagctac

SEQ ID NO: 7 - DNA, 20 bp, synthetic construct
  source 1..20: mol_type = other DNA
ggcctaactacggctacact

SEQ ID NO: 8 - DNA, 25 bp, synthetic construct
  source 1..25: mol_type = other DNA
ctctgctgaagccagttaccttcgg

SEQ ID NO: 9 - DNA, 23 bp, synthetic construct
  source 1..23: mol_type = other DNA
cgcaatcacgaatgaataacggt

SEQ ID NO: 10 - DNA, 21 bp, synthetic construct
  source 1..21: mol_type = other DNA
ccagacttgttcaacaggcca

SEQ ID NO: 11 - DNA, 26 bp, synthetic construct
  source 1..26: mol_type = other DNA
acgctcgtcatcaaaatcactcgcat